(19) United States
(12) Patent Application Publication
(10) Pub. No.: US 2024/0044823 A1
(43) Pub. Date: Feb. 8, 2024

(54) METHOD AND SYSTEM FOR QUANTITATIVE EVALUATION OF SEALING PROPERTY OF SHALE SYSTEM

(71) Applicant: China University of Petroleum (East China), Qingdao (CN)

(72) Inventors: Yuxin HAO, Qingdao (CN); Shang XU, Qingdao (CN); Qiyang GOU, Qingdao (CN)

(73) Assignee: China University of Petroleum (East China), Qingdao (CN)

(21) Appl. No.: 18/478,709

(22) Filed: Sep. 29, 2023

Related U.S. Application Data
(63) Continuation of application No. PCT/CN2022/131402, filed on Nov. 11, 2022.

(30) Foreign Application Priority Data
Jan. 21, 2022 (CN) 2022100743422

(51) Int. Cl. G01N 23/2251; G01N 15/08; G01N 33/24

(52) U.S. Cl. CPC G01N 23/2251; G01N 15/088; G01N 33/24; H01J 37/222

(57) ABSTRACT

The present invention relates to the technical field of unconventional oil and gas geology. It discloses a method and a system for quantitatively evaluating the sealing property of shale systems. Utilizing optical microscopy and field emission scanning electron microscopy, the present invention quantifies a bitumen content and pore structure parameters in a shale reservoir following qualitative identification. It classifies shale formations into four sealing types: early-stage closed and late-stage closed, early-stage closed and late-stage open, early-stage open and late-stage closed, and early-stage open and late-stage open. This classification is of significance for accurately evaluating shale preservation conditions and exploration potential. The present invention verifies the evaluations of sealing property of the shale systems by virtue of the measured formation pressures and the actual production data from shale gas wells, and is conferred with improved scientific rigor by judgment and comparison.

### CROSS-REFERENCE TO RELATED APPLICATIONS

The application claims priority to Chinese patent application No. 2022100743422, filed on Feb. 21, 2022, the entire contents of which are incorporated herein by reference.

### TECHNICAL FIELD

The present invention relates to the technical field of unconventional oil and gas geology, in particular to a method and a system for quantitative evaluation of the sealing property of a shale system.

### BACKGROUND

At present, in the complex geological evolution process the sealing property of a shale system directly affects the oil/gas-bearing property of a shale reservoir. In most cases, where a shale system has a good sealing property, a large amount of oil and gas molecules aggregate to form deposits in a shale reservoir and overpressure is developed; organic matter pores have a large pore size and a regular morphology under the support of fluid pressure; and a high-yield shale gas flow is generally generated after crushing, embodied by overpressure and rich gas. Conversely, where a shale system has a poor sealing property, the massive loss of hydrocarbon substances causes the entire strata to be at normal pressure or negative pressure; organic matter pores collapse, deform or close due to the lack of fluid pressure support; and the shale gas yield after crushing is low, embodied by gas bearing at normal pressure or lean gas at negative pressure. In general, quantitative evaluation of the sealing property of shale systems provides a scientific basis for the efficient exploration and exploitation of shale gas.

Currently scholars in China and overseas commonly evaluate the sealing property of shale systems qualitatively based on indexes, such as a fault-fracture development rate and a formation pressure. However, in practical applications these indexes are subject to uncertainty. For example, where shale fractures are relatively developed, fracture-type shale gas deposits may be formed due to good conditions of the shale roof and floor plates or because shale itself is cracked but not broken, and the shale system is still in good sealing conditions. In addition, a high formation pressure may come from in-situ stress rather than overpressure caused by pressurization during hydrocarbon generation in shale.

Through the foregoing analysis, the problems and defects in the prior art include uncertainties in the existing methods for qualitatively evaluating the sealing property of shale systems based on indexes, e.g., a fault-fracture development rate or a formation pressure.

The difficulties in solving the above problems and defects lie in accurate prediction of the development rate and the formation period of faults-fractures at different scales, while faults-fractures and formation pressure are indexes for indirectly evaluating the sealing property of a shale system.

The meaning of solving the above problems and defects is that the sealing property of a shale system could be evaluated directly, accurately and quantitatively.

### SUMMARY

For the existing problems in the prior art, the present invention provides a method and a system for quantitatively evaluating the sealing property of a shale system, and in particular relates to a method and a system for evaluating the sealing property of an unconventional shale system using an optical microscope and a scanning electron microscope to solve the problems of uncertainties in the existing evaluation indexes.

The present invention is realized using a method for quantitatively evaluating the sealing property of a shale system, comprising:
Step 1: evaluating the early-stage sealing capacity of a shale system;
Step 2: evaluating the late-stage sealing capacity of the shale system; and
Step 3: evaluating the overall sealing capacity of the shale system.

Further, the Step 1 of evaluating the early-stage sealing capacity of the shale system comprises:
(1) qualitatively identifying a bitumen content under an optical microscope and making a qualitative judgment, including direct observation through reflected light and observation under oil immersion conditions; wherein under the optical microscope, bitumen is typically filled among mineral particles and present in off-white or creamy-white; and
(2) counting the development rate of organic matters on a two-dimensional plane in a low-magnification image captured by a high-resolution scanning electron microscope; dividing the low-magnification image into several regions in proper size and zooming in these regions one by one; identifying and quantitatively counting the kerogen content according to the differences in pores in the organic matters; and calculating the plane content of bitumen in the two-dimensional image.

Further, the organic matters include bitumen and kerogen, and the kerogen and the bitumen are identified according to the following standards:
(1) Kerogen is an organic matter that has been originally deposited and never migrated, and is closely associated with terrigenous minerals; bitumen is an organic matter having flow properties produced by hydrocarbon generation in the middle and late stages of diagenesis, and is often found in residual intergranular pores, fossil cavities, and intragranular pores formed by later corrosion, at the periphery of which authigenic minerals are often seen;
(2) Pores in kerogen are seldom developed or substantially not developed, and developed pores have a directional arrangement; when not affected by external conditions such as structural damage, pores in bitumen are in a relatively regular morphology characterized by the shape of a sponge or bubble in an unoriented structure and a uniform and large pore size.

Further, more than 15 to 20 viewsheds are observed under the background of low magnification, so that the distribution characteristics of the identified organic matters could represent the actual conditions of shale samples as much as possible, and the plane content of bitumen is an average value of all viewsheds. Combined with the observation and statistical results of bitumen under the optical microscope and the scanning electron microscope, shale systems are preliminarily divided into an early-stage closed type and an early-stage open type.

Further, the Step 2 of evaluating the late-stage sealing capacity of the shale system comprises:

The late-stage sealing capacity of the shale system refers to the preservation condition in the later structural uplift process after the shale reaches the peak of gas generation in the maximum burial depth. The late-stage sealing capacity of the shale system is evaluated based on the bitumen pore characteristics under a scanning electron microscope. When the shale system has a good sealing capacity in the late stage, early-stage residual hydrocarbon substances generate gas and are pressurized and preserved by pyrolysis; bitumen pores are in the regular shape of an oval or nearly circle under the support of overpressure, and have a relatively large pore size. Conversely, when the shale system has a poor sealing capacity in the late stage, gas is lost; overpressure is released; and bitumen pores are compressed and deformed by the compaction of the overlying strata, and have a small or undeveloped pore size. Based on the observation and analysis results of organic matter pores under the scanning electron microscope, shale systems may be divided into a late-stage closed type and a late-stage open type.

Further, the Step 3 of evaluating the overall sealing capacity of the shale system comprises:

The early-stage closed, early-stage open, late-stage closed and late-stage open types as described herein may be combined in pairs to form four different types of shale sealing systems, namely an early-stage closed and late-stage closed type, an early-stage closed and late-stage open type, an early-stage open and late-stage closed type, and an early-stage open and late-stage open type. Moreover, in combination with the formation pressure in shale and data of shale gas production, the sealing property of the shale system is comprehensively evaluated.

Another object of the present invention is to provide a system of using the method for quantitatively evaluating the sealing property of a shale system, comprising:
an early-stage sealing capability evaluation module, for evaluating the early-stage sealing capability of the shale system;
a late-stage sealing capability evaluation module, for evaluating the late-stage sealing capability of the shale system; and
an overall sealing capability evaluation module, for evaluating the overall sealing capability of the shale system.

Another object of the present invention is to provide a computer device, comprising a memory and a processor; wherein the memory stores a computer program. When the computer program is executed by the processor, the processor is allowed to execute the method for quantitatively evaluating the sealing property of a shale system.

Another object of the present invention is to provide a computer-readable storage medium storing a computer program. When the computer program is executed by a processor, the processor is allowed to execute the method for quantitatively evaluating the sealing property of a shale system.

Another object of the present invention is to provide an information and data processing terminal, which is used for realizing the system for quantitatively evaluating the sealing property of a shale system.

Combining all the above-mentioned technical solutions, the present invention achieves the following advantages and positive effects: the method for quantitatively evaluating the sealing property of a shale system provided by the present invention identifies the retained hydrocarbon content and the pore development rate in shale of different sealing systems by virtue of an optical microscope and a high-precision field emission scanning electron microscope, and establishes a method for quantitatively evaluating the sealing property of a shale system; wherein quantitative statistics of a bitumen content, quantitative characterization of pore parameters, and quantitative evaluation of the sealing property of the shale system are the keys to the present invention.

Based on optical microscopy and high-precision field emission scanning electron microscopy, the present invention quantitatively identifies the contents of kerogen and retained bitumen in shale, precisely counts pore structure parameters of shale, and establishes a method for quantitatively evaluating the sealing property of a shale system, thereby solving the problems of uncertainties in the existing evaluation indexes.

Using an optical microscope and a high-precision field emission scanning electron microscope, the present invention quantitatively counts a bitumen content and pore structure parameters in a shale reservoir on the basis of qualitative identification, establishes a method for quantitatively evaluating the sealing property of a shale system, and classifies shale formations into four different shale sealing types: an early-stage closed and late-stage closed type, an early-stage closed and late-stage open type, an early-stage open and late-stage closed type, and an early-stage open and late-stage open type, which are of great significance for accurately evaluating shale preservation conditions and exploration potential. In addition, the present invention verifies the evaluations of sealing property of the shale systems by virtue of the measured formation pressures and the actual production data from shale gas wells, and is conferred with improved scientific rigor by judgment and comparison.

The present invention has been successfully applied in Wufeng Formation-Longmaxi Formation shale in Jiaoshiba area, Sichuan Basin. The shale system in Well JYA in the main area of Jiaoshiba shows a high bitumen content, a high development rate of bitumen pores, and a regular pore morphology, belonging to an early-stage closed and late-stage closed shale system; the shale system in Well JYB in the Zilichang block of Jiaoshiba shows a high bitumen content, but a low development rate of bitumen pores, a small pore size and pores mostly in an angular shape, belonging to an early-stage closed and late-stage open shale system; the shale system in Well JYC in the Pingqiao block of Jiaoshiba shows a low bitumen content, but a well development rate of bitumen pores, determined as an early-stage open and late-stage closed shale system; the shale system in Well JYD in the Baima block of Jiaoshiba shows a low bitumen content, a low development rate of bitumen pores, and a small pore size, belonging to an early-stage open and late-stage open shale system (FIG. 7 and FIG. 8).

In order to verify reliability and applicability of the present invention, the aforementioned shale systems are evaluated in terms of formation pressure and gas-bearing properties. The results show that the shale system (early-stage closed and late-stage closed) in Well JYA has a formation pressure of up to 1.55 and a gas output of more than 200,000 cubic meters per day; the shale system (early-stage open and late-stage closed) in Well JYC has a formation pressure of up to 1.59 and a gas output of about 320,000 cubic meters per day; the shale systems in Well JYB (early-stage closed and late-stage open) and Well JYD (early-stage open and late-stage open) are both at negative pressure, and have a gas output of less than 5,000 cubic meters per day. In summary, the method for evaluating the sealing property of a shale system established by the present invention is highly reliable and applicable, and provides a scientific basis and evaluation method for efficient exploration and exploitation of shale gas.

### BRIEF DESCRIPTION OF DRAWINGS

To describe the technical solutions in the examples of the present invention more clearly, the accompanying drawings required for describing the examples are illustrated briefly below. Apparently, the accompanying drawings in the following description show merely some examples of the present invention, and a person of ordinary skill in the art may still derive other drawings from these accompanying drawings without inventive efforts.

FIG. 1 is a flowchart illustrating a method for quantitatively evaluating the sealing property of a shale system provided by an embodiment of the present invention.

FIG. 2 is a diagram showing the theory of the method for quantitatively evaluating the sealing property of a shale system provided by the embodiment of the present invention.

FIG. 3 is a structure diagram illustrating a system for quantitatively evaluating the sealing property of a shale system provided by an embodiment of the present invention.

In this diagram: 1. an early-stage sealing capability evaluation module; 2. a late-stage sealing capability evaluation module; 3. an overall sealing capability evaluation module.

FIG. 4A, FIG. 4B, FIG. 4C and FIG. 4D show the qualitative discrimination of bitumen content using oil immersion and single polarized light under optical microscope.

FIG. 4A and FIG. 4B show high bitumen content, indicating that the shale reservoir may be an early closed shale system.

FIG. 4C and FIG. 4D show low bitumen content, indicating that the shale reservoir may be an early open shale system.

FIG. 5A, FIG. 5B, FIG. 5C and FIG. 5D show images of the quantitative statistics of the bitumen content using a scanning electron microscope provided by the examples of the present invention.

FIG. 5A and FIG. 5B are images of the quantitative statistics of the contents of the organic matters (including bitumen and kerogen) under the background of low magnification provided by the examples of the present invention.

FIG. 5C and FIG. 5D are images showing identification and quantitative statistics of kerogen after zooming in FIG. 5A and FIG. 5B provided by the examples of the present invention.

FIG. 6 shows images of quantitative statistics of the pore development rate in the organic matters provided by the examples of the present invention.

FIG. 7A, FIG. 7B, FIG. 7C, FIG. 7D, FIG. 7E, FIG. 7F, FIG. 7G and FIG. 7H show images of the bitumen content and the pore development rate of shale in different wells of the research area provided by the examples of the present invention.

FIG. 7A and FIG. 7B are images of the shale system in Well JYA provided by the examples of the present invention, demonstrating a high content of retained bitumen and a high development rate of organic matter pores.

FIG. 7C and FIG. 7D are images of the shale system in Well JYB provided by the examples of the present invention, demonstrating a high content of retained bitumen and a low development rate of organic matter pores.

FIG. 7E and FIG. 7F are images of the shale system in Well JYC provided by the examples of the present invention, demonstrating a low content of retained bitumen and a high development rate of organic matter pores.

FIG. 7G and FIG. 7H are images of the shale system in Well JYD provided by the examples of the present invention, demonstrating a low content of retained bitumen and a low development rate of organic matter pores.

FIG. 8 shows diagrams of classification of the shale systems in the research area provided by the examples of the present invention.

### DETAILED DESCRIPTION OF THE EMBODIMENTS

To illustrate the objects, technical solutions and advantages of the present invention more clearly, the present invention is further described in detail below with reference to examples. It should be understood that the specific examples as described herein are merely used to explain but not construed to limit the present invention.

For the existing problems in the prior art, the present invention provides a method and a system for quantitatively evaluating the sealing property of a shale system. The invention present is described in detail with reference to the accompanying drawings.

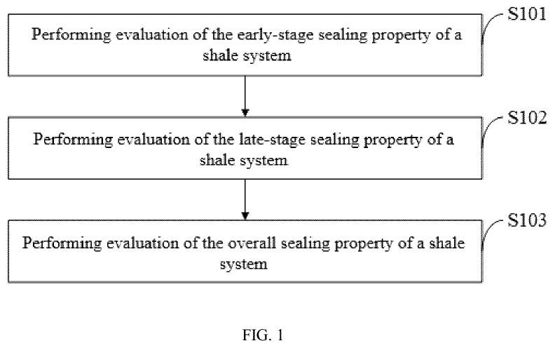

As shown in FIG. 1, a method for quantitatively evaluating the sealing property of a shale system provided by an example of the present invention comprises:
Step 101: evaluating the early-stage sealing capacity of a shale system;
Step 102: evaluating the late-stage sealing capacity of the shale system; and
Step 103: evaluating the overall sealing capacity of the shale system.

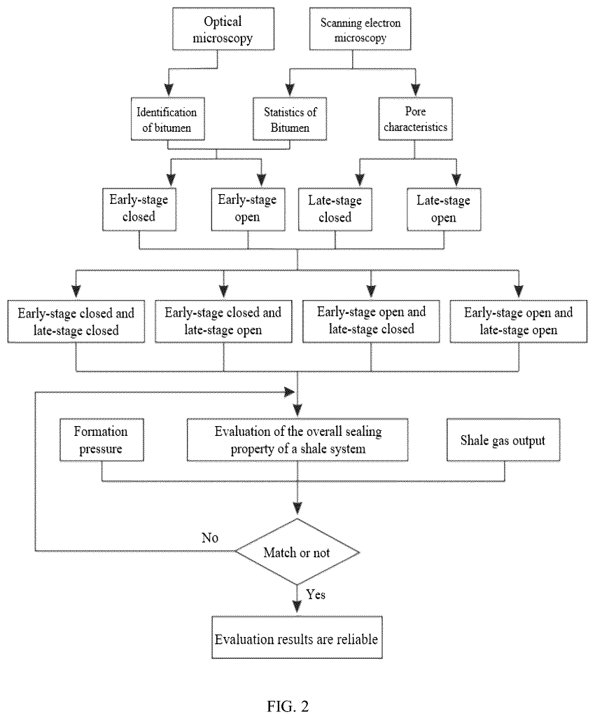

The theory of the method for quantitatively evaluating the sealing property of a shale system provided in the example of the present invention is as shown in FIG. 2.

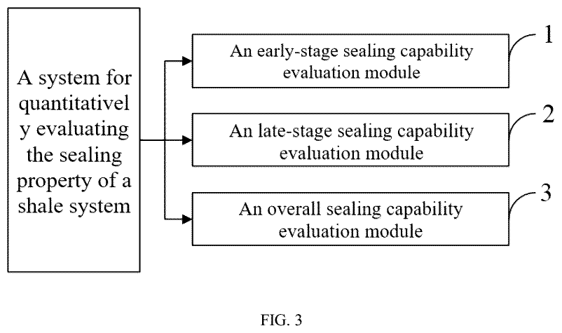

As shown in FIG. 3, a system for quantitatively evaluating the sealing property of a shale system provided by an example of the present invention comprises:
an early-stage sealing capability evaluation module 1, for evaluating the early-stage sealing capability of the shale system;
a late-stage sealing capability evaluation module 2, for evaluating the late-stage sealing capability of the shale system; and
an overall sealing capability evaluation module 3, for evaluating the overall sealing capability of the shale system.

The technical solutions of the present application are further described with reference to the following specific examples.

1. Outline of the Invention

In view of uncertainties in the existing indexes when evaluating the sealing property of a shale system, the present invention identifies the retained hydrocarbon content and the pore development rate in shale of different sealing systems by virtue of an optical microscope and a high-precision field emission scanning electron microscope, and establishes a method for quantitatively evaluating the sealing property of a shale system.

Based on optical microscopy and high-precision field emission scanning electron microscopy, the present invention quantitatively identifies the contents of kerogen and retained bitumen in shale, precisely counts pore structure parameters of shale, in an attempt to establish a method for quantitatively evaluating the sealing property of a shale system, thereby solving the problems of uncertainties in the existing evaluation indexes.

The technical solution of the present invention is shown in FIG. 2. Specifically, quantitative statistics of a bitumen content, quantitative characterization of pore parameters, and quantitative evaluation of the sealing property of a shale system are the keys to the present invention.

2. Aspects of the Invention

2.1 Evaluation of the Early-Stage Sealing Capacity of a Shale System

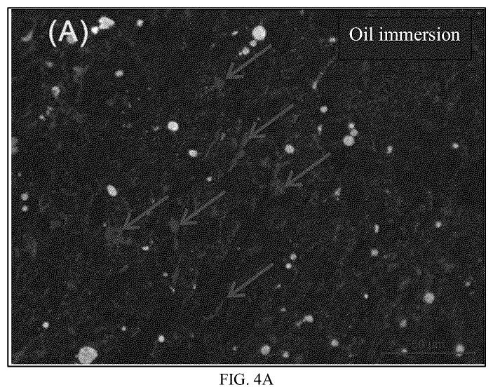

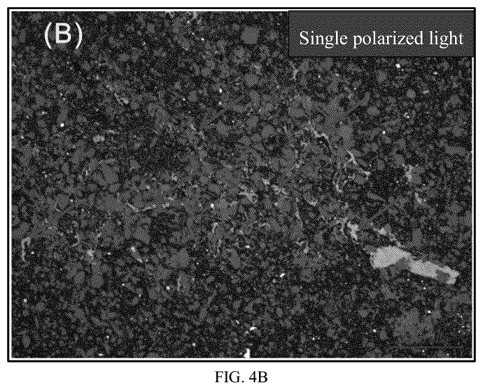

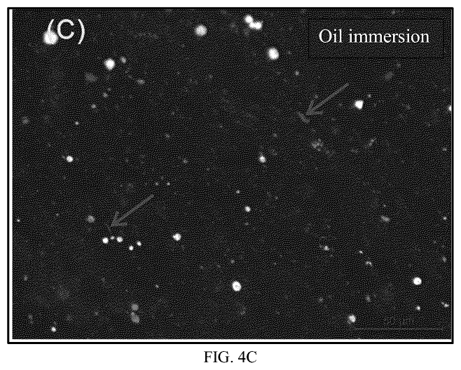

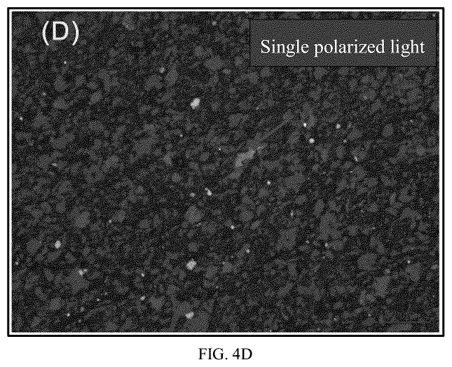

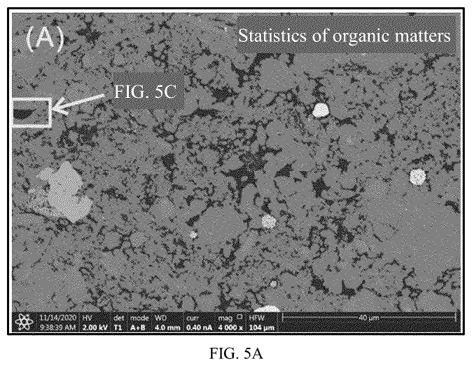

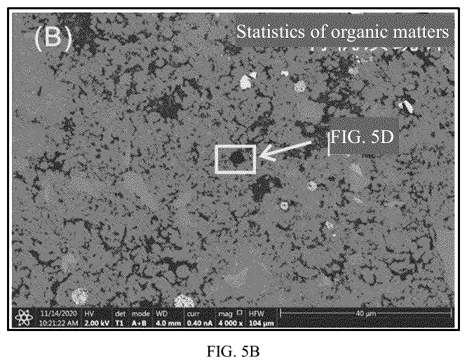

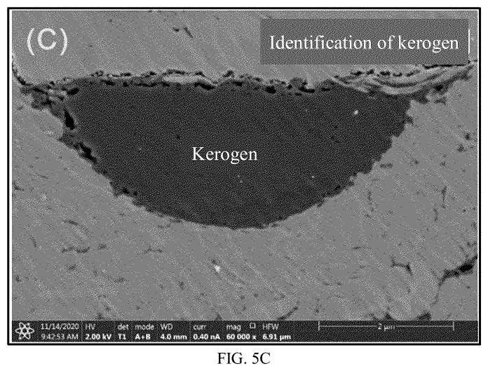

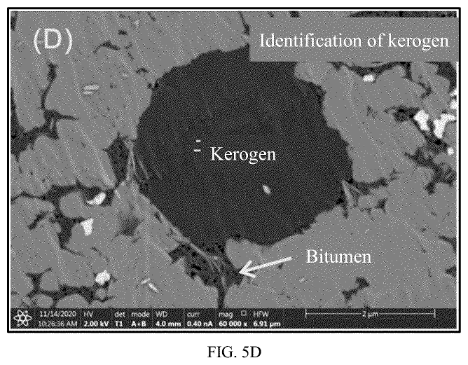

A bitumen content is an important parameter reflecting the sealing property of a shale system in the oil generation stage (i.e., an early stage). Generally speaking, the higher a bitumen content, the better the sealing property of a shale system in the oil generation stage. Evaluation of the early-stage sealing capacity of a shale system herein may be divided into two stages. The first stage is to qualitatively identify the bitumen content under an optical microscope, including direct observation through reflected light and observation under oil immersion conditions (FIG. 4). Under the optical microscope, bitumen is typically filled among mineral particles and present in off-white or creamy-white. It is worth noting that since kerogen and bitumen have similar characteristics under an optical microscope, they are hardly distinguished. As such, this result is only a qualitative judgment, and further observation using an electron microscope is also required. The second stage is to count the development rate of organic matters (including bitumen and kerogen) on a two-dimensional plane in a low-magnification image (e.g., 4000×) captured by a high-resolution scanning electron microscope. Further, the low-magnification image is divided into several regions in proper size and these regions are zoomed in one by one; the kerogen content is identified and quantitatively counted according to the differences in pores in the organic matters, and the plane content of bitumen in the two-dimensional image is further calculated (FIG. 5). Kerogen and bitumen are identified according to the following standards: (1) kerogen is an organic matter that has been originally deposited and never migrated, and is closely associated with terrigenous minerals; bitumen is an organic matter having flow properties produced by hydrocarbon generation in the middle and late stages of diagenesis, and is often found in residual intergranular pores, fossil cavities, and intragranular pores formed by later corrosion, at the periphery of which authigenic minerals are often seen; and (2) pores in kerogen are seldom developed or substantially not developed, and developed pores have a directional arrangement (FIG. 5); when not affected by external conditions such as structural damage, pores in bitumen are in a relatively regular morphology characterized by the shape of a sponge or bubble in an unoriented structure and a uniform and large pore size. In order to improve reliability of statistical results, it should be guaranteed that more than 15 to 20 viewsheds are observed under the background of low magnification, so that the distribution characteristics of the identified organic matters could represent the actual conditions of shale samples as much as possible, and the plane content of bitumen is an average value of all viewsheds. Combined with the observation and statistical results of bitumen under the optical microscope and the scanning electron microscope, the shale systems may be preliminarily divided into an early-stage closed type and an early-stage open type.

2.2 Evaluation of the Late-Stage Sealing Capacity of Shale System

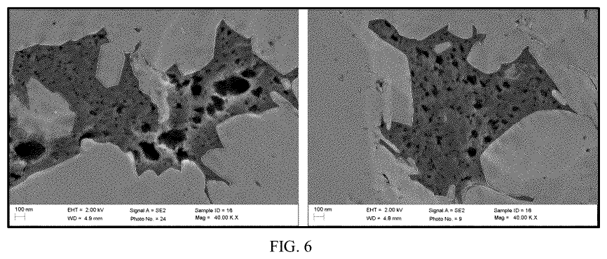

The late-stage sealing capacity of the shale system refers to the preservation condition in the later structural uplift process after the shale reaches the peak of gas generation in the maximum burial depth. The late-stage sealing capacity of a shale system is evaluated primarily based on the bitumen pore characteristics under a scanning electron microscope. Where a shale system has a good sealing capacity in the late stage, early-stage residual hydrocarbon substances generate gas and are pressurized and preserved by pyrolysis; bitumen pores are in the regular shape of an oval or nearly circle under the support of overpressure, and have a relatively large pore size. Conversely, where a shale system has a poor sealing capacity in the late stage, gas is lost; overpressure is released; and bitumen pores are compressed and deformed by the compaction of the overlying strata, and have a small or undeveloped pore size. Based on the observation and analysis results of organic matter pores under the scanning electron microscope (FIG. 6), the shale systems may be divided into a late-stage closed type and a late-stage open type.

2.3 Evaluation of the Overall Sealing Capacity of a Shale System

The early-stage closed, early-stage open, late-stage closed and late-stage open types as described herein may be combined in pairs to form four different types of shale sealing systems, namely an early-stage closed and late-stage closed type, an early-stage closed and late-stage open type, an early-stage open and late-stage closed type, and an early-stage open and late-stage open type. Moreover, in combination with formation pressure in shale and data of shale gas production, the sealing property of shale systems is comprehensively evaluated.

3. Positive Effects of the Present Invention

Using an optical microscope and a high-precision field emission scanning electron microscope, the present invention quantitatively counts a bitumen content and pore structure parameters in a shale reservoir on the basis of qualitative identification, establishes a method for quantitatively evaluating the sealing property of a shale system, and classifies shale formations into four different shale sealing types: an early-stage closed and late-stage closed type, an early-stage closed and late-stage open type, an early-stage open and late-stage closed type, and an early-stage open and late-stage open type, which are of great significance for accurately evaluating shale preservation conditions and exploration potential. In addition, the present invention verifies the evaluations of sealing property of the shale systems by virtue of the measured formation pressures and the actual production data from shale gas wells, and is conferred with improved scientific rigor by judgment and comparison.

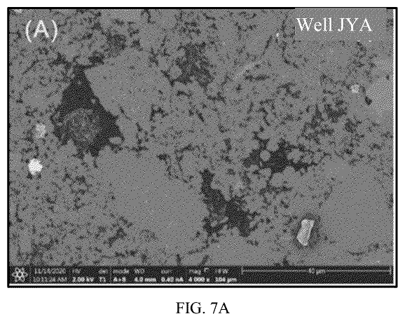

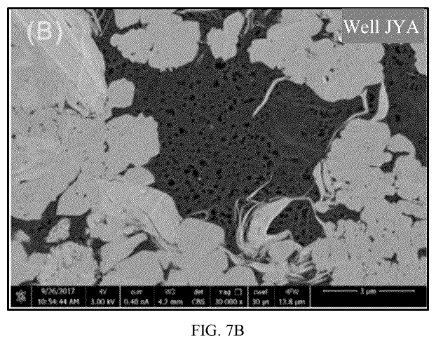

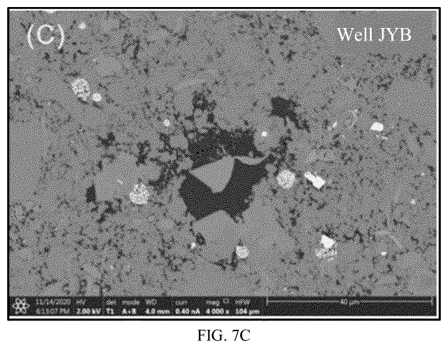

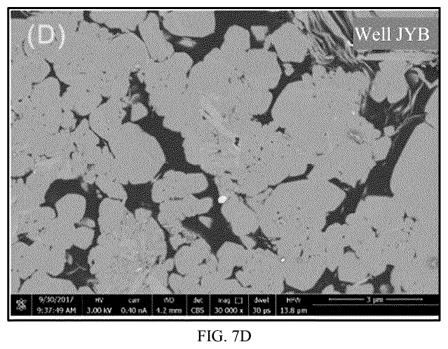

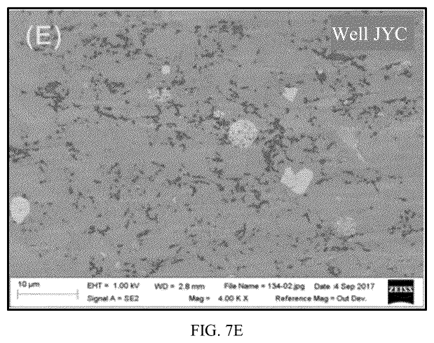

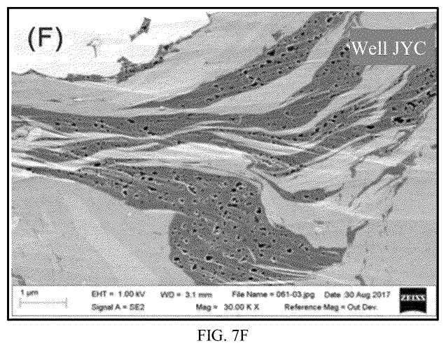

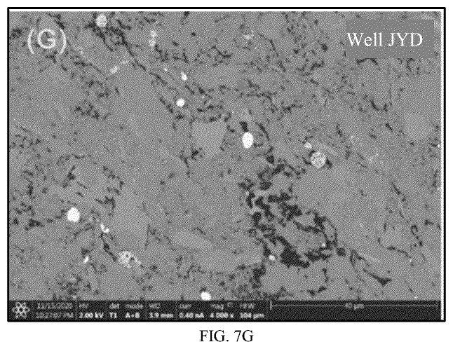

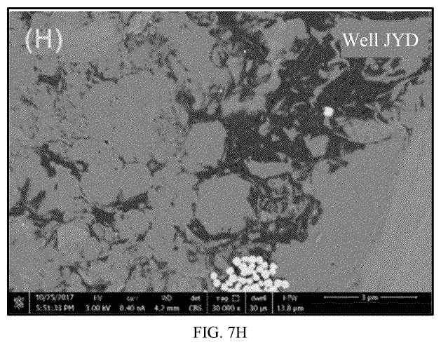

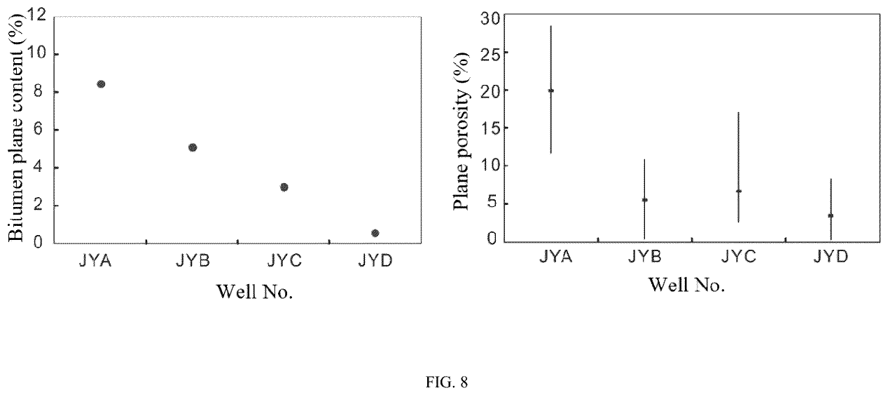

The present invention has been successfully applied in Wufeng Formation-Longmaxi Formation shale in Jiaoshiba area, Sichuan Basin. The shale system in Well JYA in the main area of Jiaoshiba shows a high bitumen content, a high development rate of bitumen pores, and a regular pore morphology, belonging to an early-stage closed and late-stage closed shale system; the shale system in Well JYB in the Zilichang block of Jiaoshiba shows a high bitumen content, but a low development rate of bitumen pores, a small pore size and pores mostly in an angular shape, belonging to an early-stage closed and late-stage open shale system; the shale system in Well JYC in the Pingqiao block of Jiaoshiba shows a low bitumen content, but a well development rate of bitumen pores, determined as an early-stage open and late-stage closed shale system; the shale system in Well JYD in the Baima block of Jiaoshiba shows a low bitumen content, a low development rate of bitumen pores, and a small pore size, belonging to an early-stage open and late-stage open shale system (FIG. 7 and FIG. 8).

In order to verify reliability and applicability of the present invention, the aforementioned shale systems are evaluated in terms of formation pressure and gas-bearing properties. The results show that the shale system (early-stage closed and late-stage closed) in Well JYA has a formation pressure of up to 1.55 and a gas output of more than 200,000 cubic meters per day; the shale system (early-stage open and late-stage closed) in Well JYC has a formation pressure of up to 1.59 and a gas output of about 320,000 cubic meters per day; the shale systems in Well JYB (early-stage closed and late-stage open) and Well JYD (early-stage open and late-stage open) are both at negative pressure, and have a gas output of less than 5,000 cubic meters per day. In summary, the method for evaluating the sealing property of a shale system established by the present invention is highly reliable and practicable, and provides a scientific basis and evaluation method for efficient exploration and exploitation of shale gas.

4. Technical Keys Claimed in the Present Invention

Evaluation of the early-stage sealing capacity of a shale system (2.1), evaluation of the late-stage sealing capacity of a shale system (2.2), evaluation of the overall sealing capacity of a shale system (2.3) and the flowchart of quantitatively evaluating the sealing property of a shale system (FIG. 2).

5. Explanation of Drawings

FIG. 4 shows qualitative judgment of the bitumen content in shale using oil immersion and single polarized light under an optical microscope. The high bitumen content in FIG. 4A and FIG. 4B indicates that the shale system may be an early-stage closed shale system; the low bitumen content in FIG. 4C and FIG. 4D indicates that the shale system may be an early-stage open shale system.

FIG. 5 shows the quantitative statistics of the bitumen content under a scanning electron microscope. FIG. 5A and FIG. 5B are images showing the quantitative statistics of the content of organic matters (including bitumen and kerogen) under the background of low magnification; FIG. 5C and FIG. 5D are images showing identification and quantitative statistics of kerogen after zooming FIG. 5A and FIG. 5B in. The differences between the plane content of organic matter in FIG. 5A/FIG. 5B and the kerogen content in FIG. 5C/FIG. 5D may be used to quantitatively identify the bitumen plane content of shale samples.

FIG. 6 shows quantitative statistics of the pore development rate in organic matters. Yellow circles the boundaries of organic matters, blue circles the boundaries of matrix minerals, and red circles the boundaries of organic matter pores. The plane porosity of organic matters (i.e., the development rate of pores in organic matters) may be represented by an area of the red zone/(an area of the yellow zone−an area of the blue zone)*100%.

FIG. 7 compares the bitumen content and the pore development rate of shale in different wells in the research area. FIG. 7A and FIG. 7B: a shale system in Well JYA with a high content of retained bitumen and a high development rate of organic matter pores; FIG. 7C and FIG. 7D: a shale system in Well JYB with a high content of retained bitumen and a low development rate of organic matter pores; FIG. 7E and FIG. 7F: a shale system in Well JYC with a low content of retained bitumen and a high development rate of organic matter pores; FIG. 7G and FIG. 7H: a shale system in Well JYD with a low content of retained bitumen and a low development rate of organic matter pores.

FIG. 8 relates to the classification of the shale systems in the research area. The shale in Well JYA is in an early-stage closed and late-stage closed system having a formation pressure of up to 1.55 and a gas output of more than 200,000 cubic meters per day; the shale in Well JYB is in an early-stage closed and late-stage open system having a formation pressure of 0.95 and a gas output of less than 5,000 cubic meters per day; the shale in Well JYC is in an early-stage open and late-stage closed system having a formation pressure of up to 1.59 and a gas output of about 320,000 cubic meters per day; the shale in Well JYD is in an early-stage open and late-stage open system having a formation pressure of 0.9 and a gas output of less than 5,000 cubic meters per day.

In the description of the present invention, unless otherwise stated, “a plurality of” means two or more than two; orientations or positional relationships indicated by terms “upper,” “lower,” “left,” “right,” “inner,” “outer,” “front end,” “rear end,” “head,” “tail,” or the like are based on the orientations or positional relationships shown in the accompanying drawings. These terms are presented merely for facilitating describing the present invention and simplifying the description, and do not indicate or imply that a device or element that is referred to must have a specific orientation or is constructed and operated in a specific orientation. Therefore, they should not be construed as limitations to the present invention. In addition, the terms “first”, “second” and “third” are merely intended for a purpose of description and shall not be understood as an indication or implication of relative importance.

All or some of the foregoing embodiments may be implemented by using software, hardware, firmware, or any combination thereof. Where all or some of embodiments may be implemented in a form of a computer program product, the computer program product includes one or more computer instructions. Where the computer program instructions are loaded and executed on a computer, the procedures or functions are all or partially generated according to embodiments of the present application. The computer may be a general-purpose computer, a special-purpose computer, a computer network, or another programmable apparatus. The computer instructions may be stored in a computer-readable storage medium or may be transmitted from one computer-readable storage medium to another computer-readable storage medium. For example, the computer instructions may be transmitted from a web site, computer, server, or data center to another web site, computer, server, or data center in a wired (for example, a coaxial cable, an optical fiber, or a digital subscriber line (DSL)) or wireless (for example, infrared, radio, or microwave) manner. The computer-readable storage medium may be any usable medium accessible to a computer, or a data storage device integrating one or more usable media, such as a server or a data center. The usable medium may be a magnetic medium (for example, a floppy disk, a hard disk, or a magnetic tape), an optical medium (for example, a DVD), a semiconductor medium (for example, a solid-state drive (SSD)), or the like.

The above descriptions are merely specific embodiments of the present invention, and shall not be construed as limitation to the protection scope of the present invention. Hence, any amendments, equivalent replacements or improvements made in the spirit and principle of the present invention by a person of ordinary skill in the art within the protection scope disclosed in the present invention shall be covered within the protection scope of the present invention.

What is claimed is:

1. A method for quantitatively evaluating the sealing property of a shale system, comprising:
Step 1: evaluating the early-stage sealing capacity of a shale system;
Step 2: evaluating the late-stage sealing capacity of the shale system; and
Step 3: evaluating the overall sealing capacity of the shale system;
wherein the Step 1 of evaluating the early-stage sealing capacity of the shale system comprises:
(1) qualitatively identifying a bitumen content under an optical microscope and making a qualitative judgment, including direct observation through reflected light and observation under oil immersion conditions; wherein under the optical microscope, bitumen is typically filled among mineral particles and present in off-white or creamy-white; and
(2) counting the development rate of organic matters on a two-dimensional plane in a low-magnification image captured by a high-resolution scanning electron microscope; dividing the low-magnification image into several regions in proper size and zooming in these regions one by one; identifying and quantitatively counting the kerogen content according to the differences in pores in organic matters; and calculating the plane content of bitumen in the two-dimensional image;
wherein the organic matters include bitumen and kerogen, and the kerogen and the bitumen are identified according to the following standards:
(1) kerogen is an organic matter that has been originally deposited and never migrated, and is closely associated with terrigenous minerals; bitumen is an organic matter having flow properties produced by hydrocarbon generation in the middle and late stages of diagenesis, and is often found in residual intergranular pores, fossil cavities, and intragranular pores formed by later corrosion, at the periphery of which authigenic minerals are often seen;
(2) pores in kerogen are seldom developed or substantially not developed, and developed pores have a directional arrangement; when not affected by structural damage under external conditions, pores in bitumen are in a relatively regular morphology characterized by the shape of a sponge or bubble in an unoriented structure and a uniform and large pore size;
wherein more than 15 to 20 viewsheds are observed under the background of low magnification, so that the distribution characteristics of the identified organic matters represent the actual conditions of shale samples as much as possible, and the plane content of bitumen is an average value of all viewsheds; combined with the observation and statistical results of bitumen under the optical microscope and the scanning electron microscope, the shale system is preliminarily classified as an early-stage closed type or an early-stage open type;
wherein the Step 2 of evaluating the late-stage sealing capacity of the shale system comprises:
the late-stage sealing capacity of the shale system refers to the preservation condition in the later structural uplift process after the shale reaches the peak of gas generation in the maximum burial depth; the late-stage sealing capacity of a shale system is evaluated based on the bitumen pore characteristics under a scanning electron microscope; when the shale system has a good sealing capacity in the late stage, early-stage residual hydrocarbon substances generate gas and are pressurized and preserved by pyrolysis, and bitumen pores are in the regular shape of an oval or nearly circle under the support of overpressure and have a relatively large pore size; conversely, when the shale system has a poor sealing capacity in the late stage, gas is lost, overpressure is released, and bitumen pores are compressed and deformed by the compaction of the overlying strata in a small or undeveloped pore size; based on the observation and analysis results of organic matter pores under the scanning electron microscope, the shale system is classified as a late-stage closed type or a late-stage open type;
wherein that the Step 3 of evaluating the overall sealing capacity of the shale system comprises:
the early-stage closed, early-stage open, late-stage closed and late-stage open types are combined in pairs to form four different types of shale sealing systems, namely an early-stage closed and late-stage closed type, an early-stage closed and late-stage open type, an early-stage open and late-stage closed type, and an early-stage open and late-stage open type; and in combination with formation pressure in shale and data of shale gas production, the sealing property of the shale system is comprehensively evaluated.

2. A system of using the method for quantitatively evaluating the sealing property of a shale system according to claim 1, comprising:
an early-stage sealing capability evaluation module, for evaluating the early-stage sealing capability of the shale system;
a late-stage sealing capability evaluation module, for evaluating the late-stage sealing capability of the shale system; and
an overall sealing capability evaluation module, for evaluating the overall sealing capability of the shale system.

3. A computer device, comprising a memory and a processor; wherein the memory stores a computer program; when the computer program is executed by the processor, the processor is allowed to execute the method for quantitatively evaluating the sealing property of a shale system according to claim 1.
